(19) United States
(12) Patent Application Publication
(10) Pub. No.: US 2004/0147441 A1
(43) Pub. Date: Jul. 29, 2004

(54) Methods and reagents for preventing bacteremias

(76) Inventors: Timothy S. Leach, Groton, MA (US); Jeffrey Packman, Weston, MA (US)

Correspondence Address: CLARK & ELBING LLP, 101 FEDERAL STREET, BOSTON, MA, 02110, US

(21) Appl. No.: 10/650,099

(22) Filed: Aug. 25, 2003

Related U.S. Application Data
(60) Provisional application No. 60/405,800, filed on Aug. 23, 2002.

(51) Int. Cl. A61K 38/17; A61K 31/704; A61K 31/65; A61K 31/545; A61K 31/397; A61K 31/407

(52) U.S. Cl. 514/12; 514/37; 514/192; 514/200; 514/253.08; 514/312; 514/152; 514/210.09

(57) ABSTRACT

The present invention provides methods and compositions useful for preventing bacteremia by decolonizing the intestinal tract of a patient. Although the present invention is useful for preventing bacteremia by any Gram-positive bacteria, it is particularly useful against antibiotic-resistant bacteria, such as vancomycin-resistant Enterococcus (VRE), methicillin-resistant Staphylococcus aureus (MRSA), glycopeptide intermediary susceptible Staphylococcus aureus (GISA), and penicillin-resistant Streptococcus pneumoniae (PRSP). Decolonization therapy using the methods and compositions of this invention are also useful for preventing a Gram-negative bacteremia.

### CROSS-REFERENCE TO RELATED APPLICATIONS

This application claims benefit of the filing date of the copending U.S. Provisional Application No. 60/405,800 (filed Aug. 23, 2002), hereby incorporated by reference.

### BACKGROUND OF THE INVENTION

This invention relates to the field of mammalian bacterial infections.

Gram-positive bacteria are becoming an important cause of nosocomial infection. The most common pathogenic isolates in hospitals include Enterococcus faecalis, Enterococcus faecium, Staphylococcus aureus, and Streptococcus pneumoniae, many strains of which are resistant to one or more antibiotics.

Enterococcus spp. are part of the normal gut flora in humans. Of the more than seventeen enterococcal species, only E. faecalis and E. faecium commonly colonize and infect humans in detectable numbers (E. faecalis is isolated from approximately 80% of human infections, and E. faecium from most of the rest). Enterococci account for approximately 25,000 cases of bacteremia annually in the United States, with most infections occurring in hospitals. Attributable mortality due to enterococcal infection deaths have also been difficult to ascertain because severe comorbid illnesses are common; however, enterococcal sepsis is implicated in 7% to 50% of fatal cases.

Vancomycin-resistant enterococcus (VRE) spp. are becoming increasingly common in hospital settings. In the first half of 1999, 25.9% of entercoccal isolates from Intensive Care Units were vancomycin-resistant; an increase from 16.6% in 1996 and from 0.4% in 1989. VRE are commonly resistant to many commercial antibiotics, including beta-lactams and aminoglycosides. Thus, patients who are immunocompromised or those having a prolonged hospital stay are at increased risk for acquiring a VRE infection. Several case-control and historical cohort studies show that death risk associated with antibiotic-resistant enterococcal bacteremia is several fold higher than death risk associated with susceptible enterococcal bacteremia.

The problem of antibiotic resistance is not unique to Entercoccus spp. Strains of many other potentially pathogenic Gram-positive bacteria displaying antibiotic resistance have been isolated including methicillin-resistant Staphylococcus aureus (MRSA), glycopeptide intermediate-susceptible Staphylococcus aureus (GISA), vancomycin-resistant MRSA (VR-MRSA) and penicillin-resistant Streptococcus pneumoniae (PRSP). Like VRE, therapeutic options for treating infections by these organisms are limited.

Resistance transfer is another complicating factor in the management of antibiotic-resistant infections. Enterococcus, for example, exhibits at least three phenotypes of vancomycin resistance: VanA—high level resistance to vancomycin and teicoplanin, VanB—moderate level resistance to vancomycin but susceptibility to teicoplanin, and VanC—low level resistance to vancomycin but susceptibility to teicoplanin. Vancomycin resistance can transfer from VRE to other Gram-positive bacteria, including S. aureus, in vitro. Therefore, the presence of VRE in a hospital poses not just the risk of VRE infections but also of continuing evolution of resistance, possibly involving more virulent organisms.

Despite the development of a plethora of new antibiotics, there is a need for new methods for treating or preventing bacteremia caused by resistant gastrointestinal bacterial flora and other Gram-positive bacteria such as VRE.

### SUMMARY OF THE INVENTION

We have discovered that blood infections in patients whose intestinal tracts are colonized by either Gram-positive bacteria, Gram-negative bacteria, or both may be prevented by substantially decolonizing the intestinal tracts by orally administering an effective amount of one or more of the compounds or members of the classes of compounds provided in Table 1.

While intestinal decolonization therapy may be administered to any person, its use to prevent either a Gram-positive or a Gram-negative bacteremia in patients at risk for developing such a bacteremia is particularly desirable. Patients in greatest need of decolonization therapy are those at high risk who have also been identified as having an intestinal colonization of antibiotic-resistant Gram-positive bacteria.

Accordingly, the invention features a preventive method that includes the steps of identifying a patient whose intestinal tract is colonized with Gram-positive bacteria, but who does not have a bacteremia caused by the bacteria, and orally administering to the patient one or more antibiotics selected from the group consisting of teicoplanin, daptomycin, oritavancin, dalbavancin, eveminomycin, virginiamycin, quinupristin, dalfopristin, linezolid, tigecycline, pristinamycin, nisin, moenomycin, gemifloxacin, tunicamycin, cinnamycin, laspartomycin, novobiocin, ciprofloxacin, moxifloxacin, chloramphenicol, nitrofurantoin, cyclo-(Leu-Pro), fosfomycin, telithromycin, azithromycin, magainin, iseganan, BMS-284,756, L-749,345, ER-35,786, S-4661, L-786,392, MC-02479, Pep5, RP 59500, and TD-6424, in an amount and for a duration sufficient to substantially decolonize the intestinal tract of the patient of the bacteria. In addition to these antibiotics, any antibiotic from the group consisting of glycopeptides, bacteriocins, type A lantibiotics, type B lantibiotics, liposidomycins, mureidomycins, alanoylcholines, quinolines, everninomycins, glycylcyclines, carbapenems, cephalosporins, streptogramins, oxazolidonones, tetracyclines, cyclothialidines, bioxalomycins, cationic peptides, and protegrins can also be administered in the practice of this preventative method.

The patient at risk can be identified by common clinical microbiological techniques. In one embodiment, the method further includes culturing the bacteria contained in a fecal specimen or rectal swab obtained from the patient. Alternatively, molecular techniques can be used for bacterial identification. For example, the patient can be identified by a nucleic acid analysis of bacteria isolated from the patient.

In a related method, the invention features a preventive method that includes the step of orally administering to a patient one or more antibiotics selected from the group consisting of teicoplanin, daptomycin, oritavancin, dalbavancin, eveminomycin, quinupristin/dalfopristin, linezolid, tigecycline, pristinamycin, nisin, moenomycin, gemifloxacin, tunicamycin, cinnamycin, laspartomycin, novobiocin, ciprofloxacin, moxifloxacin, chloramphenicol, nitrofurantoin, cyclo-(Leu-Pro), fosfomycin, telithromycin, azithromycin, BMS-284,756, L-749,345, ER-35,786, S-4661, L-786,392, MC-02479, Pep5, and TD-6424, in an amount and for a duration sufficient to substantially decolonize the intestinal tract of the patient of Gram-positive bacteria, wherein substantially all of the antibiotic is non-absorbable or partially non-absorbable, and retains antibacterial activity in the lumen of the patient's intestinal tract.

The methods of this invention are particularly useful for preventing bacteremias caused by antibiotic-resistant Gram-positive bacteria such as Enterococcus spp. including E. faecium, E. faecalis, E. raffinosus, E. avium, E. hirae, E. gallinarum, E. casseliflavus, E. durans, E. malodoratus, E. mundtii, E. solitarius, and E. pseudoavium; Staphylococcus spp. including S. aureus, S. epidermidis, S. hominis, S. saprophyticus, S. hemolyticus, S. capitis, S. auricularis, S. lugdenis, S. warneri, S. saccharolyticus, S. caprae, S. pasteurii, S. schleiferi, S. xylosus, S. cohnii, and S. simulans; Streptococcus spp. including S. pyogenes, S. agalactiae, S. pneumoniae, S. bovis, and viridans Streptococci, any of which can be resistant to treatment with antibiotics such as teicoplanin, daptomycin, oritavancin, dalbavancin, eveminomycin, quinupristin/dalfopristin, linezolid, tigecycline, pristinamycin, nisin, moenomycin, gemifloxacin, tunicamycin, cinnamycin, laspartomycin, novobiocin, ciprofloxacin, moxifloxacin, chloramphenicol, nitrofurantoin, cyclo-(Leu-Pro), fosfomycin, telithromycin, azithromycin, BMS-284,756, L-749,345, ER-35,786, S-4661, L-786,392, MC-02479, Pep5, or TD-6424, or one or more antibiotics selected from the group consisting of glycopeptides, eveminomycins, streptogramins, lipopeptides, oxazolidonones, bacteriocins, type A lantibiotics, type B lantibiotics, liposidomycins, mureidomycins, β-lactam antibiotics, and alanoylcholines. Specifically, intestinal decolonization therapy using the methods and compositions of the present invention are effective for preventing bacteremias caused by vancomycin-resistant Enterococcus spp. (VRE), methicillin- or glycopeptide-resistant Staphylococcus spp. (e.g., MRSA, GISA, or VR-MRSA), and penicillin-resistant Streptococcus spp. (e.g., PRSP).

In another embodiment of any of the methods of the invention, the patient is at high risk for developing Gram-positive bacteremia, especially from antibiotic-resistant bacteria. The patient may be neutropenic, within 14 days (prior or subsequent to) of receiving chemotherapy or radiation therapy in preparation for autologous or allogeneic hematopoietic stem cell transplant, bone marrow transplant or solid organ transplant, within 14 days (prior or subsequent to) of receiving antineoplastic radiation or chemotherapy, or at risk for enteritis, colitis, or mucositis of the intestinal tract.

In another embodiment, the patient is diagnosed as having a human immunodeficiency virus (HIV) infection, or has acquired immunodeficiency syndrome (AIDS). In yet another embodiment, the patient is diagnosed as having chronic renal insufficiency.

The patient may have an illness leading to hospitalization or institutionalization for at least one week, or an illness leading to hospitalization in an intensive care unit for at least three consecutive days, or may have an infection requiring broad-spectrum antibiotic administration for at least one week.

The methods and compositions described here are equally applicable for decolonizing the gastrointestinal tract of Gram-negative bacteria, thereby preventing a patient from developing a Gram-negative bacteremia. Gram-negative bacteremias, including those caused by Salmonella spp. (e.g., S. typhimurium, S. enteritidis, S. newport, S. anatum, S. typhi, S. paratyphi, S. schottmuelleri, and S. hirschteidii), Shigella spp., (e.g., S. dysenteriae, S. flexneri, S. boydii, and S. sonnet), pathogenic Escherichia spp., Yersinia spp. (e.g., Y. enterocolitica and Y. pestis), Proteus spp. (e.g., P. mirabilis and P. vulgaris, Klebsiella pneumoniae, and members of the Vibrionaceae family including, for example, Vibrio cholerae, Campylobacter jejuni, may be prevented by intestinal decolonization therapy.

A patient “at risk” for developing a Gram-positive bacteremia is defined as any patient who is colonized with Gram-positive bacteria. The Gram-positive bacteria that colonizes an “at risk” patient may have normal antibiotic sensitivity, intermediate (reduced) antibiotic sensitivity, or the bacteria may be antibiotic-resistant.

A patient at “high risk” is defined as a patient who is colonized with Gram-positive bacteria and who has a condition, or is undergoing or will undergo a medical therapy, that compromises or impairs their immune system. The Gram-positive bacteria that colonizes a “high risk” patient may have normal antibiotic sensitivity, intermediate (reduced) antibiotic sensitivity, or the bacteria may be antibiotic-resistant.

By “patient” is meant any human in need of medical treatment. For the purposes of this invention, patients are typically institutionalized in a primary medical care facility such as a hospital or nursing home. However, antibiotic therapy for depopulating the intestinal tract of antibiotic-resistant Gram-positive bacteria can occur on an out-patient basis, upon discharge from a primary care facility, or can be prescribed by a physician (e.g., general practitioner) for home-care, not in association with a primary medical care facility.

By “antibiotic-resistant Gram-positive bacteria” is meant any Gram-positive bacteria that have reduced (partially or completely) susceptibility to one or more antibiotics. Antibiotic classes to which Gram-positive bacteria develop resistance include, for example, the penicillins (e.g., penicillin G, ampicillin, methicillin, oxacillin, and amoxicillin), the cephalosporins (e.g., cefazolin, cefuroxime, cefotaxime, and ceftriaxone, ceftazidime), the carbapenems (e.g., imipenem, ertapenem, and meropenem), the tetracyclines and glycylclines (e.g., doxycycline, minocycline, tetracycline, and tigecycline), the aminoglycosides (e.g., amikacin, gentamycin, kanamycin, neomycin, streptomycin, and tobramycin), the macrolides (e.g., azithromycin, clarithromycin, and erythromycin), the quinolones and fluoroquinolones (e.g., gatifloxacin, moxifloxacin, sitafloxacin, ciprofloxacin, lomefloxacin, levofloxacin, and norfloxacin), the glycopeptides (e.g., vancomycin, teicoplanin, dalbavancin, and oritavancin), dihydrofolate reductase inhibitors (e.g., cotrimoxazole, trimethoprim, and fusidic acid), the streptogramins (e.g., synercid), the oxazolidinones (e.g., linezolid) and the lipopeptides (e.g., daptomycin).

“Colonized” or “colonization,” as used herein, refers to a population of bacteria in the intestinal tract that is present in the intestinal tract, but does not cause disease. The population of the intestinal tract by normal intestinal flora, as described herein, is exemplary of what is meant by colonization.

By “substantially decolonize” is meant to reduce the population of competent target bacteria in the intestinal tract by at least two log units, as determined by the quantification of bacterial growth from a fecal sample, or to reduce the population to undetectable levels from a rectal swab. Each of these determinations can be performed using standard microbiological techniques, such as those that conform to the standards provided by the American Society for Microbiology (Manual of Clinical Microbiology (7^{th} ed.) eds. Murray P R, Barron E J, Pfaller M A, Tenover F C, and Yolken R H, 1999, American Society for Microbiology, Washington). Most desirably, complete decolonization results in a reduction of the competent population of target bacteria to levels that are undetectable by standard microbiological culture methods. Decolonization can also include the eradication or suppression of the bacteria.

By “decolonization therapy” is meant a regimen for administration of an antibiotic from Table 1 in an amount and duration sufficient to substantially decolonize the intestinal tract of a patient of Gram-positive bacteria (e.g., antibiotic-resistant Gram-positive bacteria). Preferably, decolonization therapy is provided prior to, during, and subsequent to the risk period for infection. Desirably, decolonization therapy is provided by maintaining the amount of antibiotic in the stool of the patient at a concentration greater than the MIC for the bacteria that is the target of the therapy. Preferably, the antibiotic concentration in the stool is maintained at twice, three times, four times, five times, or higher multiple of the MIC for the target bacteria.

“Bacteremia” is defined as the presence of viable bacteria in the bloodstream of a host (e.g., a patient), detectable using standard aerobic or anaerobic cultures of the blood. A patient having a bacteremia may be symptomatic or pre-symptomatic.

“Non-absorbable” is defined as an antibiotic formulation which, when administered orally, has an absolute bioavailability of less than 10%.

By “partially non-absorbable,” when referring to an antibiotic, is meant an antibiotic formulation which, when administered orally, results in an absolute bioavailability of between 10% and 90%.

By “retains antibacterial activity” refers to a non-absorbable or partially non-absorbable antibiotic formulation which is at least 50%, 60%, 70%, 80%, 90%, 95%, or 99% bactericidal or bacteriostatic as a formulation of the same antibiotic that is more absorbable in the intestinal tract.

“Bioavailability” is defined as the fraction (F) of the orally administered dose that reaches the systemic circulation (Oates J A, Wilkinson G R. Priniciples of drug therapy, In Harrison's Principle of Internal Medicine (14^{th} ed.) 1998, McGraw Hill, New York.

### DETAILED DESCRIPTION

The present invention stems from our discovery that oral administration of the antibiotics shown in Table 1, alone or in combination with any other antibiotic, can prevent a Gram-positive bacteremia in a patient whose intestinal tract is colonized by such bacteria. Specifically, this invention is useful for preventing the development of bacteremia, in an uninfected patient, who has intestinal colonization by antibiotic-resistant Gram-positive bacteria.

Patients that are particularly vulnerable to blood-borne infection are those that are immunocompromised. Conditions that compromise the immune system include disorders and diseases such as malignancy, neutropenia, HIV infection or AIDS, or other viral or parasitic infections, chronic renal insufficiency, cirrhosis, alcoholism, extremes of age, connective tissue disorders, malnutrition, diabetes, splenectomy, sickle cell anemia, or concurrent administration of corticosteriods, immunosuppressants, or cytotoxic drugs. Patients with malignancies are also at high risk for bacteremia of gastrointestinal origin due to intestinal epithelial injury caused by chemotherapy and/or radiation therapy. Patients having a compromised barrier function of the intestinal tract are also at elevated risk for developing a bacteremia by bacteria that colonize their intestinal tract. Such conditions include patients receiving antineoplastic chemotherapy or radiation therapy, and those suffering antibiotic-induced colitis, and Crohn's disease. Most importantly, recipients of high dose chemotherapy followed by autologous or allogeneic hematopoietic stem cell transplant or bone marrow transplant or those diagnosed as having hematologic malignancies may require decolonization therapy during their treatment and recovery periods.

Included among therapies that make a patient high risk for developing a Gram-positive bacteremia are lengthy periods of hospitalization, especially in intensive care-units (ICUs), and high dose chemotherapy followed by autologous or allogeneic hematopoietic stem cell transplant or bone marrow transplant or solid organ transplants. Hospitalization for as little as one day, two days, or three days in an ICU can result in colonization of the intestinal tract with antibiotic-resistant Gram-positive bacteria, eventually resulting in a bacteremia caused by the colonization. Other medical therapies that result in immune system compromise include, for example, antineoplastic chemotherapy and radiation therapy, as well as the use of immuno-suppressive medications. Therapies that also cause a patient to be at “high risk” for developing an antibiotic-resistant Gram-positive bacteremia include prior or concomitant antibacterial therapy using vancomycin or an antibiotic with anaerobic bacterial activity.

In patients where the elevated risk of developing a bacteremia is a result of a medical procedure or treatment (e.g., antineoplastic chemotherapy), it is preferable that antibiotic therapy to substantially decolonize the intestinal tract begin at least 1 day, 3 days, 7 days, or 14 days prior to the medical procedure or treatment. In one embodiment, decolonization proceeds concomitantly with the medical procedure. If desirable, the decolonization therapy may be continued for at least 1 day, 3 days, 7 days, or 14 days subsequent to the medical procedure.

In preferred embodiments, 70%, 80%, 90%, 95%, 99%, or 100% of the antibiotic used to decolonize the intestinal tract of the patient is not absorbed into the bloodstream. Preferably, an antibiotic that has an absolute bioavailability following oral administration of less than 1%, 2%, 5%, 10%, 15%, 20%, 25%, 30%, 40%, 50%, or 60% is used for decolonization therapy. The absolute oral bioavailability of antibiotics may be reduced using oral formulations that reduce or prevent absorption of the antibiotic from the intestinal tract.

One skilled in the art would appreciate that the antibiotics listed in Table 1 is not meant to be limiting but to show a sample of antibiotics that can be used in the prevention of bacteremia. Moreover, the particular names or designated codes may be changed or later renamed.

TABLE 1
Antibiotics and Antibiotic Classes Useful for Decolonizing the Intestinal
Tract of Gram-positive Bacteria

| Antibiotics | | Antibiotic Classes |
|---|---|---|
| Oritavancin (LY-333, 328) | Daptomycin (LY-146, 032) | Bacteriocins |
| Dalbavancin | Quinupristin/dalfopristin | Lantibiotics (Type A & B) |
| Everninomycin | Virginiamycin | Liposidomycins |
| Pristinamycin | Linezolid | Mureidomycins |
| Tigecycline (GAR-936) | Nisin | Alkanoylcholines |
| Moenomycin | Gemifloxacin (SB-265, 805) | Quinolones |
| BMS-284, 756 | Tunicamycin | Everninomycins |
| MK-806 (L-749, 345) | E-1010 (ER-35, 786) | Glycylcyclines |
| S-4661 | L-786, 392 | Carbapenems |
| MC-02479 | Pep5 | Cephalosporins |
| Cinnamycin | Laspartomycin | Streptogramins |
| Teicoplanin | Novobiocin/ciprofloxacin | Oxazolidinones |
| Moxifloxacin | Chloramphenicol | Tetracyclins |
| Nitrofurantoin | cyclo-(Leu-Pro) | Cyclothialidines |
| Fosfomycin | Telithromycin | Bioxalomycins |
| Azithromycin | TD-6424 | Cationic peptides |
| RP 59500 | Magainin | Protegrins |
| Iseganan | | |

Flora of the Intestinal Tract

Normally, in the upper gastrointestinal tract of adult humans, the esophagus contains only the bacteria swallowed with saliva and food. The acidity of the stomach contents severely limits bacterial growth with Lactobacillus spp. comprising the majority of gastric bacteria. Accordingly, the proximal small intestine has relatively limited Gram-positive flora, consisting mainly of Lactobacillus spp. and Enterococcus faecalis. Typically this region has about 10⁵-10⁷ bacteria per milliliter of luminal fluid. The distal region of the small intestine contains greater numbers of Gram-positive bacteria and other normal flora including several Gram-negative species (e.g., coliforms and Bacteroides). Generally, the bacterial population and diversity increases distally, reaching 10¹¹ bacteria per milliliter of feces in the colon with Gram-positive bacterial species including, for example, Staphylococcus spp., Enterococcus spp., Streptococcus spp., and Clostridium spp.

Under normal conditions, the natural intestinal flora prevent colonization by pathogenic bacterial species. Additionally, the normal flora stimulate the production of cross-reactive antibodies in the host animal, acting as antigens and inducing immunological responses. Host defense mechanisms are a complex set of humoral and cellular processes that prevent microorganisms from invading the body including the bloodstream. While the normal bacterial flora are generally considered non-pathogenic in healthy individuals, these same bacteria can cause life-threatening infections if given the opportunity in patients with impaired immune function. Risk factors for these opportunistic infections include advanced age, organ transplantation, cancer, HIV infection, malnutrition, and other acquired or congenital causes of immune dysfunction as described supra. Such patients are susceptible to developing bacteremia by normal intestinal bacteria.

Likewise, disorders of the intestinal tract that compromise the anatomic and physiologic barrier functions of the intestinal mucosa render a patient susceptible to developing bacteremia by intestinal bacteria. Such conditions include, for example, colitis, proctitis, enteritis, mucositis, Crohn's disease, or sepsis. Many of these types of conditions can be induced by therapies for other disease indications, for example, resulting from antineoplastic chemotherapy or radiotherapy, or antibiotic-induced colitis.

Traditionally, bacteremias caused by the intestinal flora were susceptible to standard antibiotic therapy, and were thus successfully treated with known conventional antibiotics. However, with the recent emergence of strains of antibiotic-resistant bacteria, treating bacteremias of this nature has become significantly more difficult. For example, VRE faecium may be resistant to all commercially-available antibiotics including linezolid and quinupristin/dalfopristin. Furthermore, patients with underlying malignancies who are colonized by VRE have rates of VRE bacteremia as high as 19%. Patients who develop bacterermias with VRE have longer hospital and ICU stays, high mortality, and greater health care costs than patients without VRE bacteremias. Thus, identification of agents that result in the suppression and/or elimination of VRE and other intestinal antibiotic-resistant Gram-positive bacteria could significantly reduce morbidity, mortality, and cost.

The highest concentrations of antibiotic-resistant bacteria, including vancomycin-resistant Enterococcus (VRE), methicillin-resistant Staphylococcus aureus (MRSA), glycopeptide intermediate susceptible Staphylococcus aureus (GISA), and penicillin-resistant Streptococcus pneumoniae (PRSP), are found in hospitals, nursing homes, and other facilities where antibiotics are heavily used. Unfortunately, these same locations also have the highest density of susceptible, at risk patients. Patient care may be improved and nosocomial infections may be reduced by preventing, rather than treating, bacteremias by decolonizing the intestinal tract of a patient identified with antibiotic-resistant bacteria.

Detection of Gram-Positive Bacteria

Gram-positive bacteria that colonize the intestinal tract of a patient or cause a bacteremia can be easily detected and characterized by a skilled artisan. For example, the Gram-positive bacteria that colonize the intestinal tract can be isolated, for identification and sensitivity testing, from a stool sample, rectal swab, or culture using standard microbiological techniques. Generally, stool specimens are collected in clean (not necessarily sterile), wide-mouthed containers that can be covered with a tight-fitting lid. These containers should be free of preservatives, detergents, and metal ions and contamination with urine should also be avoided.

Stool specimens should be examined and cultured as soon as possible after collection because, as the stool specimen cools, the drop in pH soon becomes sufficient to inhibit the growth of many bacterial species. Direct microscopic examination of a fecal emulsion or stained smear to evaluate the presence of fecal pathogen forms may be valuable in the differential diagnosis of certain enteric infections. A bacterial smear for staining can also be prepared. If a delay in processing is anticipated, for example if the specimen is to be sent to a distant reference laboratory, an appropriate preservative should be used. Equal quantities of a 0.033 M sodium or potassium phosphate buffer and glycerol can be used to recover pathogenic bacteria for culturing and staining purposes.

For antibiotic sensitivity testing, a small amount of fecal specimen can be added to Gram-positive or other enrichment broth for the recovery of bacterial species. Alternatively, the broth may inoculated using a rectal swab. A variety of culture media containing inhibitors to the growth of normal bowel flora allows Gram-positive species to be selected. Subcultures of either isolated or mixed Gram-positive species can be prepared using antibiotic-containing culture media.

Alternatively, Gram-positive bacteria can be identified by molecular techniques, such as nucleic acid analyses. Some molecular techniques used in clinical microbiology for the analysis of drug-resistant bacteria have been described by Fluit et al. in Clin. Micro. Reviews 14: 836-71, 2001. A real time PCR method has been described by Grisold et al. in J. Clin. Microbiol. 40: 2392-97, 2002. Nucleic acid techniques can also be used to visualize bacteria, as described in U.S. patent application Serial No. 2002/0192755 Al. The above-mentioned detection techniques can be used to analyze the bacteria present in the blood or resident in the gastrointestinal tract. A comparison of blood/non-blood bacterial colonies in a patient can determine whether the prophylactic methods of the invention should be practiced.

Pharmaceutical Formulations

Pharmaceutical compositions according to the invention may be formulated to release an antibiotic substantially immediately upon administration or at any predetermined time or time period after administration. The latter types of compositions are generally known as controlled release formulations, which include formulations that create a substantially constant concentration of the drug within the intestinal tract over an extended period of time, and formulations that have modified release characteristics based on temporal or environmental criteria.

Antibiotic-containing formulations suitable for ingestion include, for example, a pill, capsule, tablet, emulsion, solution, suspension, syrup, or soft gelatin capsule. Additionally, the pharmaceutical formulations may be designed to provide either immediate or controlled release of the antibiotic upon reaching the target site. The selection of immediate or controlled release compositions depends upon a variety of factors including the species and antibiotic susceptibility of Gram-positive bacteria being treated and the bacteriostatic/bactericidal characteristics of the therapeutics. Methods well known in the art for making formulations are found, for example, in Remington: The Science and Practice of Pharmacy (20th ed.), ed. A. R. Gennaro, 2000, Lippincott Williams & Wilkins, Philidelphia, or in Encyclopedia of Pharmaceutical Technology, eds. J. Swarbrick and J. C. Boylan, 1988-1999, Marcel Dekker, New York.

Immediate release formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, camauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the antibiotic with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice. Other useful controlled release compositions are known in the art (see, for example, U.S. Pat. Nos. 4,946,685 and 6,261,601).

Formulations which target the antibiotic release to particular regions of the intestinal tract can also be prepared. The antibiotic can be encapsulated in an enteric coating which prevents release degradation and release from occurring in the stomach, but dissolves readily in the mildly acidic or neutral pH environment of the small intestine. A formulation targeted for release of antibiotic to the colon, utilizing technologies such as time-dependent, pH-dependent, or enzymatic erosion of polymer matrix or coating can also be used.

Alternatively, a multilayer formulation having different release characteristics between the layers can be prepared. These formulations can result in the antibiotic being released in different regions of the intestinal tract. A multilayer formulation of this type may be particularly useful for maintaining a more constant antibiotic concentration throughout the length of the intestinal tract. Alternatively, if the intestinal tract is colonized with more than one Gram-positive bacterial strain, where each bacterial strain preferentially colonizes a different region of the intestinal tract, a multilayer formulation can be used to deliver different antibiotics to different intestinal regions. For example, an inner core, containing an antibiotic is prepared and encapsulated in an enteric coating. An outer antibiotic-containing layer is then added. This formulation has the advantage of releasing the antibiotic contained in the outer layer into the stomach and upper duodenum, whereas the antibiotic contained in the enterically coated core is released later. Of course, the antibiotic contained in the core need not be the same as the antibiotic contained in the outer layer.

The targeted delivery properties of the antibiotic-containing formulation may be modified by other means. For example, the antibiotic may be complexed by inclusion, ionic association, hydrogen bonding, hydrophobic bonding, or covalent bonding. In addition polymers or complexes susceptible to enzymatic or microbial lysis may also be used as a means to deliver drug.

Microsphere encapsulation of the antibiotic is another useful pharmaceutical formulation for targeted antibiotic release. The antibiotic-containing microspheres can be used alone for antibiotic delivery, or as one component of a two-stage release formulation. Suitable staged release formulations may consist of acid stable microspheres, encapsulating an antibiotic to be released later in the lower intestinal tract admixed with an immediate release formulation to deliver antibiotic to the stomach and upper duodenum.

Microspheres can be made by any appropriate method, or from any pharmaceutically acceptable material. Particularly useful are proteinoid microspheres (see, for example, U.S. Pat. Nos. 5,601,846, or 5,792,451) and PLGA-containing microspheres (see, for example, U.S. Pat. Nos. 6,235,224 or 5,672,659). Other polymers commonly used in the formation of microspheres include, for example, poly-ε-caprolactone, poly(ε-caprolactone-Co-DL-lactic acid), poly(DL-lactic acid), poly(DL-lactic acid-Co-glycolic acid) and poly(ε-caprolactone-Co-glycolic acid) (see, for example, Pitt et al., J. Pharm. Sci. 68: 1534, 1979). Microspheres can be made by procedures well known in the art including spray drying, coacervation, and emulsification (see, for example, Davis et al. Microsphere and Drug Therapy, 1984, Elsevier; Benoit et al. Biodegradable Microspheres: Advances in Production Technologies, Chapter 3, ed. Benita, S, 1996, Dekker, New York; Microencapsulation and Related Drug Processes, Ed. Deasy, 1984, Dekker, New York; and U.S. Pat. No. 6,365,187).

Liquids for Oral Administration

Powders, dispersible powders, or granules suitable for preparation of aqueous solutions or suspensions by addition of water are convenient dosage forms for oral administration. Formulation as a suspension provides the active ingredient in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

Dosages

Antibiotics are administered orally in an amount and for a duration sufficient to substantially decolonize the intestinal tract of Gram-positive bacteria. Although the exact dosage of each antibiotic useful for substantially decolonizing the intestinal tract will be different, the dosage can be easily determined by a person of ordinary skill. Typically, the amount of an antibiotic that is administered is an amount that maintains the stool concentration of the antibiotic at least equal to the MIC for the target organism. Preferably, the amount of antibiotic that is administered maintains the stool concentration equivalent to two, three, four, or more times the MIC for the target organism (see Tables 2 and 3). Thus, the particular treatment regimen may vary for each specific antibiotic and each patient, dependent upon the species and resistance pattern of the identified Gram-positive bacteria, and biological factors unique to each patient including the comorbidity, disease etiology, patient age (pediatric, adult, geriatric), and the nutritional and immune status.

The dosing regimen required to substantially decolonize the intestinal tract of Gram-positive bacteria may be determined prior to the initiation of decolonization or prophylactic therapy, and may be altered during the course of the therapy. For example, decolonization of the intestinal tract can be monitored periodically or at regular intervals to measure the patient's bacterial load and dosage or frequency of antibiotic therapy can be adjusted accordingly.

Antibiotic susceptibility information may be obtained from the same cultures used to identify patients colonized with Gram-positive bacteria. Dosing regimens may be developed from information known in the art or empirically by monitoring the efficacy of decolonization therapy periodically through the treatment period. The synthesis, formulation, and use of daptomycin and its derivatives, for example, is extensively described in Drugs R&D 3:33-39, 2002, and PCT Publication Nos. WO 01/44271, WO 01/44272, WO 01/44274, WO 01/53330, WO 01/97851, WO 02/056829, and WO 02/059145 (hereby incorporated by reference). Laspartomycin is described in PCT Publication No. WO 02/05838 (hereby incorporated by reference). Likewise, therapeutic decolonization regimens for oritavancin may be guided by information provided by Coyle et al., Antimicrob. Agents Chemother. 45: 706-9, 2001, Noviello et al., J. Antimicrob. Chemther. 48: 283-6, 2001, and Barrett, Curr. Opin. Invest. Drugs 2: 1039-44, 2001 (all of which are hereby incorporated by reference). Johnston et al., (Curr. Drug Targets, 3:335-344, 2002; hereby incorporated by reference) describes many features of the streptogramin class of antibiotics. Dosages of other antibiotics useful for the practice of the invention are as recommended by the Physician 's Desk Reference, 57^{th} Edition (2003). Since many of the antibiotics currently in use have poor oral bioavailability, with elevated oral dosages required for the delivery of an effective systemic amount, it is possible that a lower dose than that which is recommended may suffice for the practice of the invention, as the methods of the present invention include the oral administration of antibiotics for treatment of bacteria resident in the gastrointestinal region. Such a lower dose can be 50%, 40%, 30%, 20%, or even 10% that of which is recommended as a suitable oral dose for systemic efficacy.

TABLE 2
In Vitro Efficacy of Selected Antibiotics Useful for Decolonizing the
Gastrointestinal Tract of Gram-positive Bacteria

| | | Efficacy Measure* |
|---|---|---|
| Organism | Antibiotic | (μg/ml) |
| S. aureus | oritavancin¹ | MIC = 2.0-4.0; MBC = 4.0-8.0 |
| (methicillin | teicoplanin¹ | MIC = 0.5-4.0; MBC = 2.0-8.0 |
| susceptible) | | |
| S. aureus | RP 59500⁵ | MIC = 0.25-0.5; MBC = 0.25-1.0 |
| (oxacillin- | | |
| susceptible) | | |
| S. aureus | oritivancin¹ | MIC = 2.0-4.0; MBC = 8.0-16.0 |
| (methicillin | teicoplanin¹ | MIC = 2.0-4.0; MBC = 8.0-16.0 |
| resistant) | | |
| S. aureus | RP 59500⁵ | MIC = 0.25-1.0; MBC = 0.25-2.0 |
| (oxacillin- | | |
| resistant) | | |
| S. pneumoniae | oritivancin¹ | MIC = <0.03; MBC = <0.03 |
| (penicillin | teicoplanin¹ | MIC = <0.03; MBC = <0.03 |
| susceptible) | | |
| S. pneumoniae | oritivancin¹ | MIC = <0.03; MBC = <0.03 |
| (penicillin | teicoplanin¹ | MIC = <0.03; MBC = <0.03 |
| resistant) | | |
| S. pyrogens | oritivancin¹ | MIC = 0.12-0.25; MBC = 0.5-1.0 |
| | teicoplanin¹ | MIC = 0.03-0.06; MBC = 0.06-0.25 |
| E. facium | oritivancin¹ | MIC = 0.12-0.25; MBC = 1.0-2.0 |
| (vancomycin | teicoplanin¹ | MIC = 0.25-0.5; MBC = 2.0-4.0 |
| susceptible) | RP 59500⁵ | MIC = 1.0; MBC = 1.0-8.0 |
| E. facium | oritivancin¹ | MIC = 1.0-2.0; MBC = 4.0-8.0 |
| (vancomycin | teicoplanin¹ | MIC = >16.0; MBC = >16.0 |
| resistant) | RP 59500⁵ | MIC = 0.5-1.0; MBC = 1.0-8.0 |
| E. facium | daptomycin² | MIC = 2.0-4.0 |
| (multidrug | teicoplanin² | MIC 8.0-1,024 |
| resistant) | | |
| E. faecalis | oritivancin¹ | MIC = 0.5-1.0; MBC = 1.0-2.0 |
| (vancomycin | teicoplanin¹ | MIC = >0.25; MBC = 2.0-4.0 |
| susceptible) | RP 59500⁵ | MIC = 8.0-16.0; MBC = 16.0-32.0 |
| E. faecalis | RP 59500⁵ | MIC = 4.0-16.0; MBC = 32.0 |
| (vancomycin | | |
| resistant) | | |
| E. faecalis | Cyclo(leu-pro)⁴ | MIC = 12.5 |
| (strains K-99-34, | | |
| K-00-184, | | |
| and K-00-221) | | |
| C. Difficile | daptomycin³ | MIC = 16.0 |

*MIC = minimal growth inhibitory concentration; MBC = minimal bactericidal concentration
¹S. Voviello et al. J. Antimicrob. Chemother. 48: 283-286, 2001.
²N. Mobarakai et al. Antimicrob. Agents Chemother. 38: 385-387, 1994.
³M. Dong et al. Antibicrob. Agents Chemother. 31: 1135-1136, 1987.
⁴R. Rhee, J. Gen. Appl. Microiol. 48: 321-327, 2002.
⁵Shonekan et al. J. Antimicrob. Chemother. 39: 405-409, 1997.

Laspartomycin core lipopeptides are particularly useful in the methods of the present invention. In vitro, the MIC₅₀ of several of these compounds has been found to be significantly increased in the presence of calcium (Table 2; also see WO 02/805838), ubiquitous in the human diet and frequently administered as a dietary supplement. Thus, oral administration of laspartomycin lipopeptides may be administered alone or in combination with calcium in order to effect intestinal decolonization of Gram-positive bacteria.

TABLE 3
Minimal Inhibitory Concentration of Laspartomycin
Derivatives Against S. aureus Strain Smith
grown in Mueller-Hinton Broth

| | MIC (no CaCl₂) | MIC (with CaCl₂) |
|---|---|---|
| | (μg/ml) | (μg/ml) |
| Daptomycin | 1 | 0.5 |
| Aspartocin | 2 | 1 |
| Zaomycin | 10 | 1 |
| Laspartomycin | 16 | 2 |

Typically, the oral dosage of an antibiotic suitable for decolonization therapy is normally at least about 0.1, 1, 2, 5, 10, or 50 mg/day up to as much as 500, 1000, 1500, 2000, or 5000 mg/day. An antibiotic may be given daily (e.g., once, twice, three times, or four times daily) or less frequently (e.g., once every other day, or once or twice weekly). The antibiotic may be contained in any appropriate amount in any suitable carrier substance, and is generally present in an amount of 1-99% by weight of the total weight of the composition. The composition is provided in a dosage form that is suitable for oral administration and delivers a therapeutically effective amount of the antibiotic to the small and large intestine, as described below.

The duration of therapy sufficient to substantially decolonize the intestinal tract of Gram-positive bacteria may also be determined on a patient-by-patient basis. Typically, therapy should last at least five days, but preferably at least one week, two weeks, three weeks, one month, two months, or more. The antibiotic therapy should at least encompass the period during which the patient is at highest risk for developing a bacteremia. More preferably, the antibiotic therapy should begin prior to, and extend beyond the patient's period of highest risk. For example, in the case of high dose chemotherapy followed by autologous or allogeneic hematopoietic stem cell transplant or bone marrow transplantation, antibiotic therapy should be started at least one week prior to the preparative chemotherapeutic regimen and continued until marrow engraftment has occurred and neutropenia has resolved. Preferably, antibiotic therapy continues for at least one or two weeks longer than the immunosuppressive therapy.

### EXAMPLE 1

### Decolonization Therapy Using Daptomycin

Prolonged hospitalization is a risk factor for intestinal colonization with multi-drug resistant Gram-positive bacteria. Bacteremias frequently result from the immunocompromised status of the patient, or other comorbidity. Of particular importance is gastrointestinal colonization (and subsequent bacteremia) with antibiotic resistant strains that are frequently present in medical institutions as a result of widespread and high dose antibiotic use. In one example, a high risk patient (e.g., a patient in the Intensive Care Unit) is monitored for the presence of antibiotic-resistant Gram-positive bacteria (i.e., VRE) in their gastrointestinal tract. Once identified, the patient is orally administered 100 mg daptomycin with 500 mg calcium twice daily (b.i.d.). Decolonization therapy is administered for seven days and the patient re-tested for the presence of the previously-identified bacteria. Therapy is continued until the fecal bacterial load is reduced by at least three logs, or is undetectable. The patient is tested for re-colonization by antibiotic-resistant Gram-positive bacteria at least once every three days following cessation of decolonization therapy. Therapy is restarted as indicated.

### Other Embodiments

All publications and patents cited in this specification are herein incorporated by reference as if each individual publication or patent was specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A preventive method comprising the steps of:
a) identifying a patient whose intestinal tract is colonized with Gram-positive bacteria, but who does not have a bacteremia caused by said bacteria; and
b) orally administering to said patient one or more antibiotics selected from the group consisting of: teicoplanin, daptomycin, oritavancin, dalbavancin, eveminomycin, virginiamycin, quinupristin, dalfopristin, linezolid, tigecycline, pristinamycin, nisin, moenomycin, gemifloxacin, tunicamycin, cinnamycin, laspartomycin, novobiocin, ciprofloxacin, moxifloxacin, chloramphenicol, nitrofurantoin, cyclo-(Leu-Pro), fosfomycin, telithromycin, azithromycin, magainin, iseganan, BMS-284,756, L-749,345, ER-35,786, S-4661, L-786,392, MC-02479, Pep5, RP 59500, and TD-6424 in an amount and for a duration sufficient to substantially decolonize the intestinal tract of said patient of said bacteria.

2. A preventive method comprising the steps of:
a) identifying a patient whose intestinal tract is colonized with Gram-positive bacteria, but who does not have a bacteremia caused by said bacteria; and
b) orally administering to said patient one or more antibiotics selected from the group consisting of bacteriocins, type A lantibiotics, type B lantibiotics, liposidomycins, mureidomycins, alanoylcholines, quinolines, eveminomycins, glycylcyclines, carbapenems, cephalosporins, streptogramins, oxazolidonones, tetracyclines, cyclothialidines, bioxalomycins, cationic peptides, and protegrins in an amount and for a duration sufficient to substantially decolonize the intestinal tract of said patient of said bacteria.

3. The method of claim 1 or 2, said method further comprising culturing said bacteria from a stool sample or rectal swab obtained from said patient.

4. The method of claim 1 or 2, said method further comprising nucleic acid analysis of said bacteria.

5. A preventive method comprising the step of orally administering to a patient one or more antibiotics selected from the group consisting of teicoplanin, daptomycin, oritavancin, dalbavancin, everninomycin, virginiamycin, quinupristin, dalfopristin, linezolid, tigecycline, pristinamycin, nisin, moenomycin, gemifloxacin, tunicamycin, cinnamycin, laspartomycin, novobiocin, ciprofloxacin, moxifloxacin, chloramphenicol, nitrofurantoin, cyclo-(Leu-Pro), fosfomycin, telithromycin, azithromycin, magainin, iseganan, BMS-284,756, L-749,345, ER-35,786, S-4661, L-786,392, MC-02479, Pep5, RP 59500, and TD-6424 in an amount and for a duration sufficient to substantially decolonize the intestinal tract of said patient of Gram-positive bacteria, wherein substantially all of said antibiotic is non-absorbable or partially non-absorbable, and retains antibacterial activity in the lumen of said intestinal tract.

6. The method of any of claims 1, 2, or 5, wherein said antibiotic is formulated such that substantially all of said antibiotic is non-absorbable or partially non-absorbable, and retains antibacterial activity in the lumen of the intestinal tract of said patient.

7. The method of any of claims 1, 2, or 5, wherein said bacteria are antibiotic-resistant.

8. The method of claim 7, wherein said antibiotic-resistant Gram-positive bacteria comprise bacteria of the genus Enterococcus.

9. The method of claim 8, wherein said bacteria are E. faecium, E. faecalis, E. raffinosus, E. avium, E. hirae, E. gallinarum, E. casseliflavus, E. durans, E. malodoratus, E. mundtii, E. solitarius, or E. pseudoavium.

10. The method of claim 9, wherein said bacteria is resistant to one or more antibiotics selected from the group consisting of vancomycin, teicoplanin, daptomycin, oritavancin, dalbavancin, eveminomycin, quinupristin/dalfopristin, linezolid, and tigecycline.

11. The method of claim 9, wherein said bacteria is resistant to one or more antibiotics selected from the group consisting of glycopeptides, eveminomycins, streptogramins, lipopeptides, oxazolidonones, bacteriocins, type A lantibiotics, type B lantibiotics, liposidomycins, mureidomycins, and alanoylcholines.

12. The method of claim 7, wherein said antibiotic-resistant Gram-positive bacteria comprise bacteria of the genus Staphylococcus.

13. The method of claim 12, wherein said bacteria is S. aureus, S. epidermidis, S. hominis, S. saprophyticus, S. hemolyticus, S. capitis, S. auricularis, S. lugdenis, S. warneri, S. saccharolyticus, S. caprae, S. pasteurii, S. schleiferi, S. xylosus, S. cohnii, or S. simulans.

14. The method of claim 13, wherein said bacteria is resistant to one or more antibiotics selected from the group consisting of methicillin, teicoplanin, daptomycin, oritavancin, dalbavancin, everninomycin, quinupristin/dalfopristin, linezolid, and tigecycline.

15. The method of claim 13, wherein said bacteria is resistant to one or more antibiotics selected from the group consisting of glycopeptides, eveminomycins, streptogramins, lipopeptides, oxazolidonones, bacteriocins, type A lantibiotics, type B lantibiotics, liposidomycins, mureidomycins, and alanoylcholines.

16. The method of claim 7, wherein said antibiotic-resistant Gram-positive bacteria comprise bacteria of the genus Streptococcus.

17. The method of claim 16, wherein said bacteria is S. pyogenes, S. agalactiae, S. pneumoniae, S. bovis, or S. viridans.

18. The method of claim 17, wherein said bacteria is resistant to one or more antibiotics selected from the group consisting of penicillin, teicoplanin, daptomycin, oritavancin, dalbavancin, eveminomycin, quinupristin/dalfopristin, linezolid, and tigecycline.

19. The method of claim 17, wherein said bacteria is resistant to one or more antibiotics selected from the group consisting of glycopeptides, everninomycins, streptogramins, lipopeptides, oxazolidonones, bacteriocins, type A lantibiotics, type B lantibiotics, liposidomycins, mureidomycins, β-lactam antibiotics, and alanoylcholines,

20. The method of any of claims 1, 2, or 5, wherein said patient is neutropenic.

21. The method of any of claims 1, 2, or 5, wherein said patient is within 14 days of receiving chemotherapy or radiation therapy in preparation for autologous or allogeneic hematopoietic stem cell transplant, bone marrow transplant or solid organ transplant.

22. The method of any of claims 1, 2, or 5, wherein said patient is within 14 days of receiving antineoplastic radiation or chemotherapy.

23. The method of any of claims 1, 2, or 5, wherein said patient has or is at risk for enteritis, colitis, or mucositis of the intestinal tract.

24. The method of any of claims 1, 2, or 5, wherein said patient is diagnosed as having a human immunodeficiency virus (HIV) infection, or has acquired immunodeficiency syndrome (AIDS).

25. The method of any of claims 1, 2, or 5, wherein said patient is diagnosed as having chronic renal insufficiency.

26. A preventive method comprising the steps of:
a) identifying a patient whose intestinal tract is colonized with Gram-negative bacteria, but who does not have a bacteremia caused by said bacteria; and
b) orally administering to said patient one or more antibiotics selected from the group consisting of: teicoplanin, daptomycin, oritavancin, dalbavancin, everninomycin, virginiamycin, quinupristin, dalfopristin, linezolid, tigecycline, pristinamycin, nisin, moenomycin, gemifloxacin, tunicamycin, cinnamycin, laspartomycin, novobiocin, ciprofloxacin, moxifloxacin, chloramphenicol, nitrofurantoin, cyclo-(Leu-Pro), fosfomycin, telithromycin, azithromycin, magainin, iseganan, BMS-284,756, L-749,345, ER-35,786, S-4661, L-786,392, MC-02479, Pep5, RP 59500, and TD-6424 in an amount and for a duration sufficient to substantially decolonize the intestinal tract of said patient of said bacteria.

27. The methods of claim 26, wherein said bacteria is selected from the group consisting of S. typhimurium, S. enteritidis, S. newport, S. anatum, S. typhi, S. paratyphi, S. schottmuelleri, S. hirschteidii, S. dysenteriae, S. flexneri, S. boydii, S. sonnei, Y. enterocolitica Y. pestis, P. mirabilis, P. vulgaris, Klebsiella pneumoniae, Vibrio cholerae, and Campylobacter jejuni.
